(19) United States
(12) Patent Application Publication
(10) Pub. No.: US 2021/0290097 A1
(43) Pub. Date: Sep. 23, 2021

(54) APPARATUS AND METHOD FOR EXTRACTING COMPONENT ASSOCIATED WITH SPECIFIC PHYSIOLOGICAL PHENOMENON BASED ON MEASUREMENT OF ELECTRIC PROPERTIES, AND RECONSTRUCTING EIT DATA BY USING EXTRACTED COMPONENT

(71) Applicant: BILAB CO., LTD., Seoul (KR)

(72) Inventor: Hun WI, Suwon-si Gyeonggi-do (KR)

(21) Appl. No.: 16/822,751

(22) Filed: Mar. 18, 2020

(51) Int. Cl. A61B 5/053; A61B 5/08; A61B 5/00

(52) U.S. Cl. CPC A61B 5/0536; A61B 5/683; A61B 5/0809

(57) ABSTRACT

An apparatus and method for extracting a component associated with the specific physiological phenomenon based on measurement of electric properties and reconstructing electrical impedance tomography (EIT) data by using the extracted component include an electrode unit comprising a plurality of electrodes to be attached to a measurement part of a subject, an electric-property measurer configured to measure a plurality of electric properties of the subject according to lapse of time through the plurality of attached electrodes, an EIT-data generator configured to generate EIT data based on change in the plurality of measured electric properties, a pattern extractor configured to extract specific pattern data associated with a specific component caused by a specific physiological phenomenon of the subject from the generated EIT-data, and an EIT-data reconstructor configured to reconstruct the generated EIT-data as EIT data corresponding to the specific component based on the extracted specific pattern data.

### CROSS-REFERENCE TO RELATED THE APPLICATION

This application is based on and claims priority under 35 U.S.C. § 119 to Korean Patent Application No. 10-2018-0121011 filed on Oct. 11, 2018 in the Korean Intellectual Property Office, the disclosure of which is incorporated by reference herein in its entirety.

### BACKGROUND

### Field

The disclosure relates to a technical concept of extracting a component associated with the specific physiological phenomenon based on measurement of electric properties and reconstructing electrical impedance tomography (EIT) data by using the extracted component, and more particularly to an apparatus and method for extracting only a component of a specific physiological phenomenon from a composite signal affected by change in internal electric properties of a human body according to a plurality of physiological phenomena based on EIT, and reconstructing EIT data by using the extracted component.

### Description of the Related Art

When voltage is measured after injecting current to or current is measured after injecting voltage to a human body or the like volume conductor, measured data may vary depending on internal electric properties such as conductivity and dielectric constant distribution, the size and shape of the volume conductor, and the position, size and shape of electrode.

Further, there is a principal component analysis (PCA) or an independent component analysis (ICA) as technology of extracting only changed components respectively corresponding to specific causes.

Here, the PCA is used as a method of reducing dimensions by representing an original signal with linear combination of principal components having high energy.

Further, the ICA refers to a calculation method of dividing a multivariate signal into statistically independent sub-components, in which the components include components statistically independent of one another as non-Gaussian signals, and the ICA may be used in blind signal division that needs only statistical independence of each signal.

However, it is not taken into account at all by the related art that the size and shape of the volume conductor and the position, shape and size of the electrode are not varied depending on time but the internal electric properties of the volume conductor are varied depending on time.

Accordingly, there is a need of proposing technology that extracts a component associated with a specific physiological phenomenon while considering change in electric properties in a state that the position and shape of an electrode is invariable with regard to a specific subject.

Further, the subject to be subjected to the EIT includes people of all ages such as an infant, a child, an adult, the aged, etc., men and women, and various races, and the size and shape of a part to be subjected to imaging may be largely varied depending generally on ages and obesity levels.

Therefore, a measurement method needs to be selected based on the obesity level, and the technology of extracting a component associated with a specific physiological phenomenon from a signal affected by many physiological phenomena has to be taken into account.

Here, the EIT technology makes an image by attaching a plurality of electrodes to a surface of an subject to be targeted at measurement, detecting impedance of the targeted subject through the plurality of electrodes, and converting the detected impedance into the conductivity and dielectric constant of the targeted subject.

The EIT technology is to produce an image of a specific internal part of a human body, and has generally been used in medical fields.

### SUMMARY

The disclosure is to extract only a component of a specific physiological phenomenon from a composite signal affected by change in internal electric properties of a human body according to a plurality of physiological phenomena.

The disclosure is to extract components respectively caused by change in air inside an upper airway, change in blood flow inside a carotid, movement of a neck at breathing, movement of a tongue, change in air inside a lung, or change in thoracic blood flow from electrical impedance tomography (EIT).

The disclosure is to restore an image based on a component associated with a specific physiological phenomenon.

The disclosure is to change a method of injecting current or voltage by considering the size and shape of a part to be subjected to imaging, and thus flexibly set a range of measuring the voltage or the current.

The disclosure is to identify an electrode pair for injecting current or voltage based on circumference of a part to be subjected to imaging, thereby flexibly setting a measurement range for voltage or current.

The disclosure is to improve quality of a restored image by increasing the number of voltages distinguishable from noise as measurement range for voltage or current is flexibly set.

According to an embodiment of the disclosure, an apparatus for extracting a specific component based on electrical impedance tomography (EIT) and reconstructing data includes: an electrode unit including a plurality of electrodes to be attached to a measurement part of a subject; an electric-property measurer configured to measure a plurality of electric properties of the subject according to lapse of time through the plurality of attached electrodes; an EIT-data generator configured to generate EIT data based on change in the plurality of measured electric properties; a pattern extractor configured to extract specific pattern data associated with a specific component caused by a specific physiological phenomenon of the subject from the generated EIT-data; and an EIT-data reconstructor configured to reconstruct the generated EIT-data as EIT data corresponding to the specific component based on the extracted specific pattern data.

The pattern extractor may analyze one of energy and frequency of the generated EIT-data through one of a signal-to-noise ratio in the generated EIT-data, principal component analysis (PCA) or independent component analysis (ICA), and extract specific pattern data associated with a specific component caused by a specific physiological phenomenon of the subject with respect to a frequency component based on the analyzed energy or frequency.

The EIT-data reconstructor may reconstruct the generated EIT-data as EIT data corresponding to the specific component, based on difference in relative voltage change level between the extracted specific pattern data and the generated EIT-data.

The apparatus for extracting a specific component based on EIT and reconstructing data according to an embodiment of the disclosure may further include an image restorer configured to restore an image associated with the specific component based on the reconstructed EIT data.

The plurality of measured electric properties may include conductivlity and dielectric constant distribution.

The specific component may include at least one of change in air inside a lung or airway of the subject, change in blood flow inside a body, change in a component inside the body, and change in movement of a part in the body.

According to an embodiment of the disclosure, an apparatus for measuring electric properties based on EIT includes: an electrode unit including a plurality of electrodes to be attached to a measurement part of a subject; and an electric-property measurer configured to measure a plurality of electric properties of the subject through the plurality of attached electrodes, the electric-property measurer identifying neighboring first and second electrodes among the plurality of electrodes as a supplying electrode pair when a circumference of the measurement part is smaller than or equal to a measurement reference value, identifying the first electrode and a third electrode positioned farther away from the first electrode than the second electrode as the supplying electrode pair when the circumference of the measurement part is greater than the measurement reference value, and identifying a plurality of supplying electrode pairs which are different in distance according to change in the circumference of the measurement part.

The electric-property measurer may supply current or voltage to the identified supplying electrode pair, and measure current or voltage induced from the current or voltage supplied through the plurality of attached electrodes.

The electric-property measurer may change a voltage gain or a current gain according to the circumference of the measurement part when the current or voltage induced from the supplied current or voltage.

According to an embodiment of the disclosure, a method of extracting a specific component and reconstructing data based on EIT includes: by an electric-property measurer, measuring a plurality of electric properties of a subject according to lapse of time through a plurality of attached electrodes attached to a measurement part of the subject; by an EIT-data generator, generating EIT data based on change in the plurality of measured electric properties; by a pattern extractor, extracting specific pattern data associated with a specific component caused by a specific physiological phenomenon of the subject from the generated EIT-data; and by an EIT-data reconstructor, reconstructing the generated EIT-data as EIT data corresponding to the specific component based on the extracted specific pattern data.

The extraction of specific pattern data associated with a specific component caused by a specific physiological phenomenon of the subject from the generated EIT-data may include analyzing one of energy and frequency of the generated EIT-data through one of a signal-to-noise ratio in the generated EIT-data, principal component analysis (PCA) and independent component analysis (ICA); and extracting specific pattern data associated with a specific component caused by a specific physiological phenomenon of the subject with respect to a frequency component based on the analyzed energy or frequency.

The reconstruction of the generated EIT-data as EIT data corresponding to the specific component based on the extracted specific pattern data may include reconstructing the generated EIT-data as EIT data corresponding to the specific component, based on difference in relative voltage change level between the extracted specific pattern data and the generated EIT-data.

According to an embodiment of the disclosure, a method of measuring electric properties based on EIT, in which the plurality of electric properties of a subject is measured through a plurality of electrodes to be attached to a measurement part of the subject may include: by an electric-property measurer, identifying neighboring first and second electrodes among the plurality of electrodes as a supplying electrode pair when a circumference of the measurement part is smaller than or equal to a measurement reference value; by an electric-property measurer, identifying the first electrode and a third electrode positioned farther away from the first electrode than the second electrode as the supplying electrode pair when the circumference of the measurement part is greater than the measurement reference value; and by an electric-property measurer, identifying a plurality of supplying electrode pairs which are different in distance according to change in the circumference of the measurement part.

According to an embodiment of the disclosure, the method of measuring electric properties based on EIT may further include: by the electric-property measurer, supplying current or voltage to the identified supplying electrode pair; measuring current or voltage induced from the current or voltage supplied through the plurality of attached electrodes; and by the electric-property measurer, changing a voltage gain or a current gain according to the circumference of the measurement part when the current or voltage induced from the supplied current or voltage.

### BRIEF DESCRIPTION OF THE DRAWINGS

FIG. 1 illustrates elements of an apparatus for extracting a specific component based on electrical impedance tomography (EIT) and reconstructing data according to an embodiment of the disclosure;

FIG. 2 illustrates an embodiment related to generation of EIT data according to an embodiment of the disclosure;

FIGS. 3A to 3G illustrate a method of extracting a specific component based on pattern data according to an embodiment of the disclosure;

FIG. 4A illustrates elements of a pattern-data processor according to an embodiment of the disclosure;

FIG. 4B illustrates an embodiment related to pattern data according to an embodiment of the disclosure;

FIG. 5 illustrates an embodiment that an image is restored by an apparatus for extracting a specific component and reconstructing data according to an embodiment of the disclosure;

FIGS. 6A and 6B illustrate embodiments related to measurement of electric properties according to an embodiment of the disclosure;

FIGS. 7 to 12 illustrate embodiments of changing combination of electrodes forming a pair for injecting current according to measured circumferences of a subject;

FIG. 13 is a flowchart related to a method of extracting a specific component and reconstructing data according to an embodiment of the disclosure; and

FIG. 14 is a flowchart related to a method of measuring electric properties according to an embodiment of the disclosure.

### DETAILED DESCRIPTION OF EXEMPLARY EMBODIMENTS

Hereinafter, various embodiments of the disclosure will be described with reference to the accompanying drawing.

Embodiments and terms used therein are not construed as limiting the disclosure to a specific form, and it will be appreciated that various changes, equivalents and/or alternative can be made in these embodiments.

In the following descriptions, details about publicly known functions or features will be omitted if it is identified that they cloud the gist of the present inventive concept.

Further, terms to be used in the following descriptions will be defined by taking functions of elements into account, but may be varied depending on a user, intent or practice of an operator, etc. Therefore, the definition needs to be given based on the content described throughout the following.

Regarding the description of the drawings, like numeral refer to like elements.

Singular forms are intended to include plural forms unless otherwise mentioned contextually.

In the disclosure, term “A or B,” “at least one of A and/or B,” or the like may include any possible combinations of items listed together.

Terms “first,” “second,” etc. may be used herein to describe elements regardless of order or importance, and only used to distinguish one element from another without limiting the elements.

It will be understood that when a certain (e.g. first) element is referred to as being “connected” or “coupled” to another (e.g. second) element (for function or communication), they can be connected or coupled each other directly or through an additional (e.g. third) element.

In this disclosure, “configured to ˜ (or set to ˜)” may for example be used interchangeably with “suitable for ˜,” “having ability to ˜,” “modified to ˜,” “made to ˜,” “capable of ˜,” or “designed to ˜” by hardware or software according to circumstances.

In some circumstances, a “device configured to ˜” may mean that the device may be “capable of ˜” together with another device or parts.

For example, a “processor configured (or set) to perform A, B, and C” may refer to an exclusive processor (e.g. an embedded processor) for performing the operations, or a universal processor (e.g. a central processing unit (CPU) or an application processor) capable of performing the operations by executing one or more software programs stored in a memory device.

Further, ‘or’ refers to ‘inclusive or’ rather than ‘exclusive or.’

In other words, unless otherwise mentioned or unless otherwise made clear from the context, ‘x uses a or b’ means one of natural inclusive permutations.

Terms ‘ . . . part’, ‘ . . . unit’ etc. to be used herein refers to a unit of processing at least one function or operation, and this may be achieved by hardware, software or combination of hardware and software.

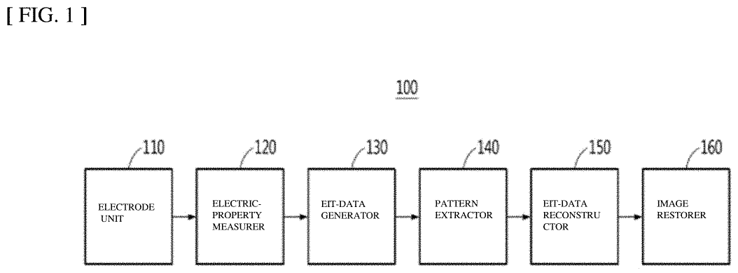

FIG. 1 illustrates elements of an apparatus for extracting a specific component based on electrical impedance tomography (EIT) and reconstructing data according to an embodiment of the disclosure.

Referring to FIG. 1, an apparatus 100 for extracting a specific component based on EIT and reconstructing data includes an electrode unit 110, an electric-property measurer 120, an EIT-data generator 130, a pattern extractor 140 and an EIT-data reconstructor 150.

According to an embodiment of the disclosure, the electrode unit 110 may include a plurality of electrodes to be attached to a measurement part of a subject.

For example, the plurality of electrodes may include at least one of a simple electrode or a combined electrode, and may include an EIT electrode to apply current and measure impedance data for voltage measurement.

For example, the EIT electrode is arranged on one side of a base plate made of a flexible elastic material and attached to the measurement part of the subject. According to an embodiment, the EIT electrode may be shaped like a belt or a vest.

Further, the EIT electrode is used in injecting current having a relatively low level, which a subject cannot feel, for example, high-frequency current having a level lower than or equal to 1 mA, and measuring induced voltage. Current-voltage data measured through the EIT electrode may be used in detecting an internal pattern of a human body through an imaging algorithm.

According to an embodiment of the disclosure, the electric-property measurer 120 measures a plurality of electric properties of the subject according to lapse of time through the plurality of electrodes attached to the subject.

For instance, the electric-property measurer 120 may identify a supplying electrode pair among the plurality of electrodes based on the circumference of the measurement part, and supply current or voltage to the supplying electrode pair. Here, the supplying electrode pair may refer to a pair of electrodes for supplying the current or the voltage.

Further, the electric-property measurer 120 may measure current or voltage induced by the current or the voltage through a measuring electrode pair among the other electrodes except the supplying electrode pair among the plurality of electrodes. Here, the measuring electrode pair may refer to a pair of electrodes for measuring the current or the voltage.

For example, the plurality of electric properties may include conductivity and dielectric constant distribution of the induced current or voltage.

According to an embodiment of the disclosure, the electric-property measurer 120 may identify first and second neighboring electrodes as the supplying electrode pair among the plurality of electrodes when the circumference of the measurement part is smaller than or equal to a measurement reference value.

For instance, the electric-property measurer 120 may identify the first electrode and a third electrode as the supplying electrode pair among the plurality of electrodes when the circumference of the measurement part is larger than the measurement reference value. Here, the third electrode may refer to an electrode relatively farther away from the first electrode than the second electrode.

According to an embodiment of the disclosure, the measurement reference value may be based on at least one among the circumference of the measurement part, the maximum or minimum measured value of the induced current or voltage, or a noise level.

For example, the measurement reference value may be set or changed by a user as s/he wants.

In addition, the measurement reference value may be used as an index for identifying a distance between the electrodes forming the pair for supplying the current.

For example, when a distance between two electrodes forming the supplying electrode pair increases, the maximum measured value and the minimum measured value may increase.

Further, when a distance between two electrodes forming the supplying electrode pair decreases, the maximum measured value and the minimum measured value may decrease.

According to an embodiment of the disclosure, the electric-property measurer 120 may measure a plurality of electric properties within a voltage measurement range calculated excluding the minimum measured value from the maximum measured value.

In other words, according to the disclosure, the method of injecting current or voltage is changed by taking the size and shape of a part to be subjected to imaging into account, and therefore the range of measuring the voltage or the current is flexibly set.

According to an embodiment of the disclosure the electric-property measurer 120 may supply the current or the voltage to the supplying electrode pair, and measure current or voltage induced by the current or the voltage supplied through the measuring electrode pair identified among the other electrodes except the supplying electrode pair among the plurality of electrodes attached to the measurement part of the subject.

For instance, the electric-property measurer 120 may measure about 208 electric properties by changing combination between the supplying electrode pair for injecting the current or the voltage and the measuring electrode pair for measuring the voltage or the current within 16 electrodes.

In more detail, the electric-property measurer 120 may generate 208 pieces of time-series data by measuring 208 electric properties for a certain period of time.

For example, the supplying electrode pair may refer to an electrode pair for supplying of the current or the voltage.

Further, the measuring electrode pair may refer to an electrode pair for measuring current or voltage induced from the supplied current or voltage.

For instance, the apparatus 100 for extracting a specific component based on EIT and reconstructing data may include an apparatus (not shown) for measuring the electric properties based on EIT, which includes the electrode unit 110 and the electric-property measurer 120.

According to an embodiment of the disclosure, the EIT-data generator 130 may generate EIT data based on change in the plurality of electric properties. Here, the generated EIT-data may be also called measured EIT data.

For instance, the EIT-data generator 130 may generate EIT data according to the voltage measurement range.

In other words, the EIT-data generator 130 may generate EIT data between the maximum measured value and the minimum measured value of the voltage.

For example, the EIT data may include change, noise, a motion artifact, etc. of the plurality of electric properties. In other words, the EIT data may be affected by impedance change associated with stenosis of an upper airway, a breathing exercise, blood flow in a carotid, and irregular movement of an underjaw and a tongue.

According to an embodiment of the disclosure, the pattern extractor 140 may use the signal-to-noise ratio of the generated EIT-data to identify at least one pieces of pattern data from the generated EIT-data.

For example, the EIT data may include a plurality of signal-to-noise ratios different from one another based on a plurality of electric properties.

In other words, the pattern extractor 140 may identify pattern data corresponding to change in 16 electric properties having a good signal-to-noise ratio among changes in 208 electric properties of the EIT data.

For example, the pattern data may also be called frequency pattern data associated with scale change of the electric properties.

Further, the pattern data may correspond to at least one piece of frequency change data according to change of the plurality of electric properties within the EIT data.

According to an embodiment of the disclosure, the pattern extractor 140 may extract pattern data corresponding to a specific component, which is caused by the physiological phenomenon of the subject, from at least one piece of pattern data.

For example, the specific component may include at least one of change in internal air of a lung or airway of a subject, change in blood flow inside a body, change in a component inside the body, and change in movement of a part in the body.

For instance, the pattern extractor 140 may use one among the signal-to-noise ratio in the EIT data, a principal component analysis (PCA) or an independent component analysis (ICA) to analyze one of the energy or the frequency in the EIT data.

Further, the pattern extractor 140 may extract specific pattern data associated with a specific component caused by a specific physiological phenomenon of a subject with respect to a frequency component based on the analyzed energy or frequency.

According to the disclosure, it is possible to extract only a component caused by a specific physiological phenomenon from a composite signal affected by change in internal electric properties of a human body according to a plurality of physiological phenomena.

In other words, according to the disclosure, it is possible to extract components respectively caused by change in air inside an upper airway, change in blood flow inside a carotid, movement of a neck at breathing, movement of a tongue, change in air inside a lung, or change in thoracic blood flow from the EIT measurement data.

According to an embodiment of the disclosure, the EIT-data reconstructor 150 may reconstruct the EIT data as EIT data corresponding to a specific component based on the extracted pattern data.

For instance, the EIT-data reconstructor 150 may reconstruct the EIT data as EIT data corresponding to a specific component based on difference in relative voltage change between the extracted specific pattern data and the EIT data.

In other words, the EIT-data reconstructor 150 may use a least square error method to rescale the EIT data because the same difference is given between relative sizes of the pattern data measured for certain periods of time.

According to an alternative embodiment of the disclosure, an apparatus 100 for extracting a specific component based on the EIT and reconstructing data may further include an image restorer 160.

According to an embodiment of the disclosure, the image restorer 160 may restore an image associated with a specific component based on the reconstructed EIT data.

For instance, the image restorer 160 may use the EIT data divided based on a specific component to restore an image associated with the specific component.

For example, when the specific component is a component caused by change in air inside a lung or change in thoracic blood flow, the image restorer 160 may individually restore an image of the change in air inside the lung or an image of the change in the thoracic blood flow.

An embodiment of individually restoring the image of the change in air inside the lung or the image of the change in the thoracic blood flow will be additionally described later with reference to FIG. 5.

According to the disclosure, it is possible to restore an image based on a component associated with a specific physiological phenomenon.

In other words, according to the disclosure, it is possible to improve quality of a restored image by increasing the number of voltages distinguishable from noise as measurement range for voltage or current is flexibly set.

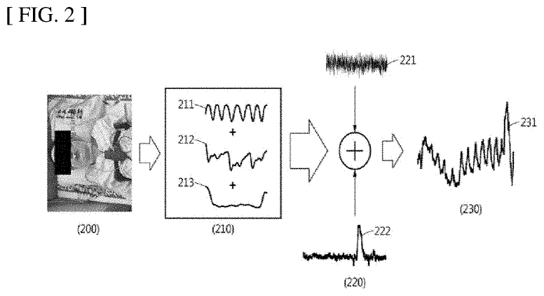

FIG. 2 illustrates an embodiment related to generation of the EIT data according to an embodiment of the disclosure.

In more detail, FIG. 2 illustrates an embodiment that EIT data is generated with regard to a neck of a subject.

At operation 200, a method of extracting a specific component and reconstructing data includes attaching a plurality of electrodes to a portion under a face of a subject.

At operation 210, the method of extracting a specific component and reconstructing data includes measuring a plurality of electric properties involving component changes such as a breathing-related motion artifact 211, blood flow 212, upper-airway occlusion 213, etc. through the plurality of electrodes attached to a portion under the face of the subject.

At operation 220 the method of extracting a specific component and reconstructing data may include measuring the plurality of electric properties of the subject according to lapse of time. Here, the plurality of electric properties to be measured may include noise 221 and noise caused by the movement of the subject.

At operation 230, the method of extracting a specific component and reconstructing data may include generating EIT data 231 based on change in the plurality of electric properties. Here, the EIT data may also be called the measured EIT data.

For example, the EIT data 231 may have been affected by impedance change associated with stenosis of an upper airway, a breathing exercise, blood flow in a carotid, and irregular movement of an underjaw and a tongue.

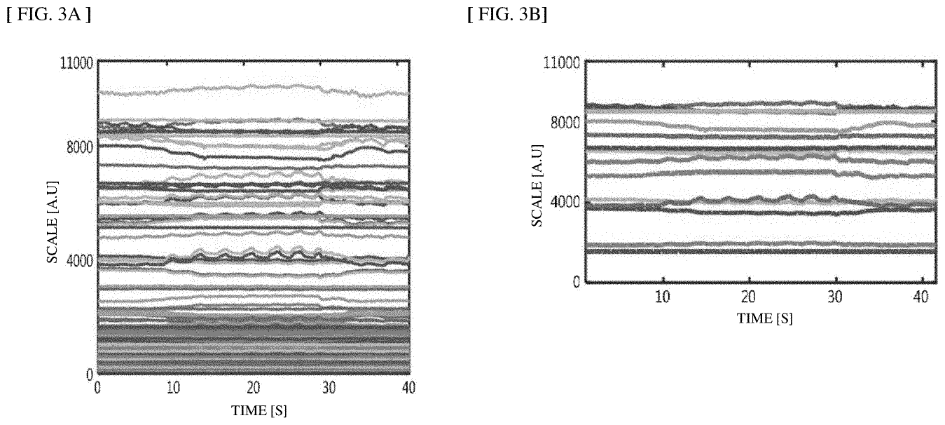

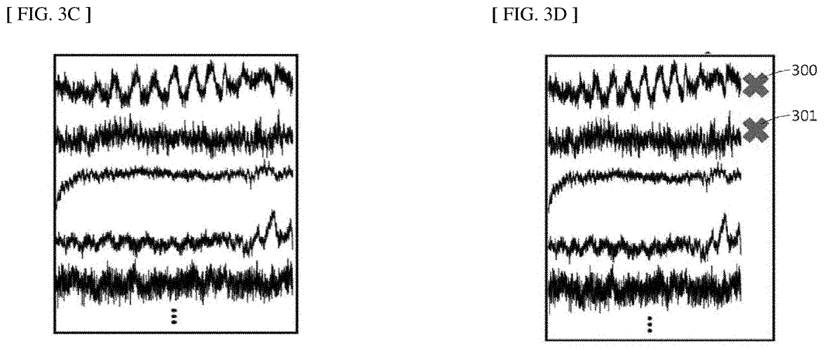

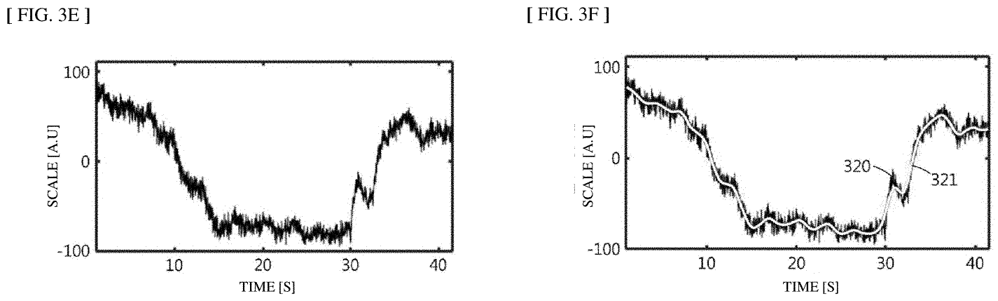

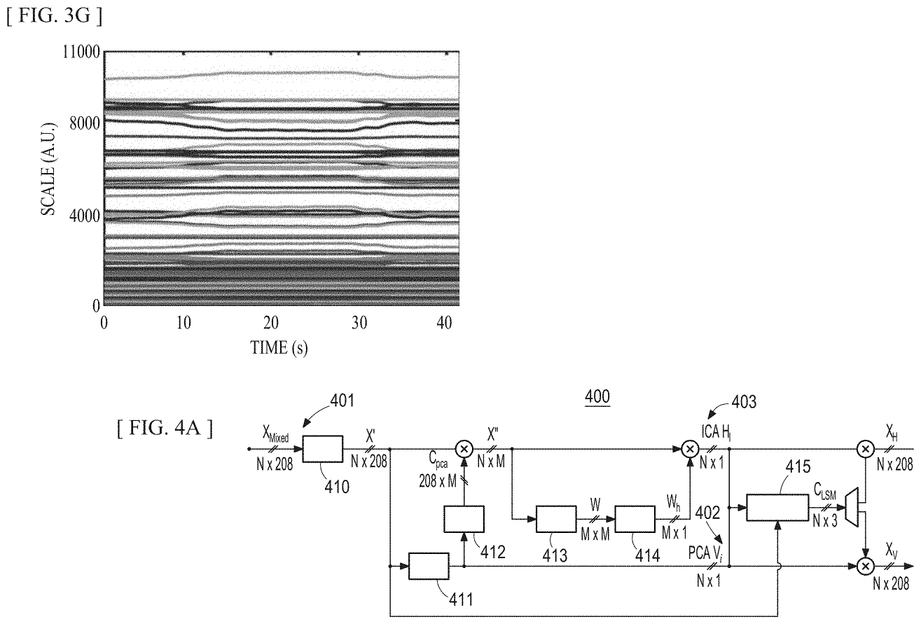

FIGS. 3A to 3G illustrate a method of extracting a specific component based on pattern data according to an embodiment of the disclosure.

FIG. 3A shows a result that the EIT-data generator according to an embodiment of the disclosure uses 16 electrodes to generate EIT data, which involves change in 208 electric properties through 208 channels. Here, the change in the electric properties may show scale change in the electric properties as time passes. Further, channels may be set based on combination of 16 electrodes.

FIG. 3B shows a result that the pattern extractor according to an embodiment of the disclosure uses the signal-to-noise ratio to identify changes of 16 electric properties having a high signal-to-noise ratio (SNR) among changes of 208 electric properties.

For instance, the pattern extractor may select 16 voltage channels, which have the highest SNR among 208 time-series voltage channels, as inputs for an ICA algorithm.

FIG. 3C shows a result that the pattern extractor according to an embodiment of the disclosure identifies pattern data corresponding to change in 16 electric properties. For example, the identified pattern data may correspond to an ICA component.

FIG. 3D shows a result that the pattern extractor according to an embodiment of the disclosure removes noise pattern data 300 and noise pattern data 301 from 16 ICA components.

For example, when an independent source signal S is calculated, breathing exercise and blood flow components may be identified through a spectrum analysis.

When the fast-Fourier transform is applied to all the independent components of the independent source signal, the breathing component having basic frequencies corresponding to a respiratory rate and a heart rate may be identified as breathing exercise and blood flow components. A modified source signal U may be calculated based on the following Expression 1.

U=Ŵ⁻¹Ŝ [Expression 1]

In [Expression 1], Ŵ⁻¹ may be a modified mixing matrix, and Ŝ may be an independent source signal.

Ŵ⁻¹ may be obtained by replacing columns, which corresponds to elements of the breathing exercise and the blood flow, with columns of 0.

FIG. 3E shows a result that the pattern extractor according to an embodiment of the disclosure extracts pattern data corresponding to a specific component 310. For example, the specific component 310 may correspond to an upper airway signal.

FIG. 3F shows a result that the pattern extractor according to an embodiment of the disclosure applies filtering to the pattern data corresponding to the specific component 310. Referring to FIG. 3F, a graph 320 corresponds to the specific component 310, and a graph 321 corresponds to the upper airway signal passed through a low pass filter.

Here, 208 pieces of voltage data corresponding to upper airway stenosis may be restored to have proper amplitude. Further, the low pass filter may be used to reduce residual noise of the restored voltage data without distorting a pattern of the upper airway stenosis.

FIG. 3G shows a result that the EIT-data reconstructor according to an embodiment of the disclosure reconstructs the EIT data including change in 208 electric properties corresponding to the specific component based on the extracted pattern data.

For example, the EIT-data reconstructor may reconstruct the EIT data including change in 208 electric properties based ono the following [Expression 2].

Vⱼ=aⱼU_{UA}+bⱼ [Expression 2]

In the Expression 2, Vⱼ may be voltage of the jth channel, U may be a modified source signal, and aⱼ and bⱼ may be constants corresponding to difference values between the pieces of the voltage data.

In addition, the EIT-data reconstructor may generate matrix data based on the following [Expression 3], and the generated matrix data may correspond to [Expression 4].

C=(U_{UA}^{T}U_{UA})⁻¹U_{UA}^{T}X [Expression 3]

In the [Expression 3], C may be the reconstructed EIT data, U may be the modified data, T may be time, and X may be the initially generated EIT-data.

$$C = {[ \begin{matrix}{a}_{1} & … & {a}_{208} \\ {b}_{1} & … & {b}_{208}\end{matrix} ]}^{T} \qquad [ Expression 4 ]$$

In the [Expression 4], C may be the reconstructed EIT data, a and b may be constants corresponding to voltage differences, and T may be time.

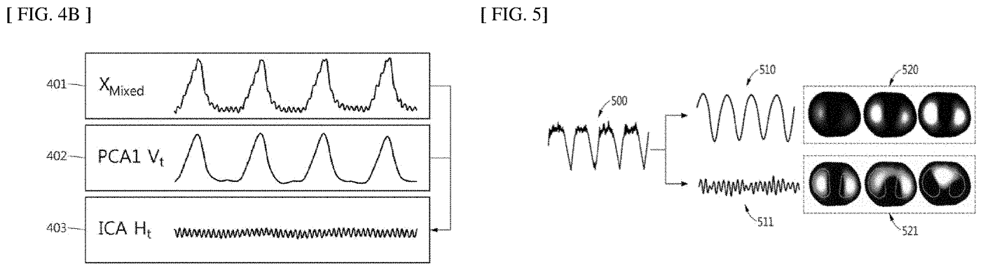

FIG. 4A illustrates elements of a pattern-data processor according to an embodiment of the disclosure.

FIG. 4A shows operation procedures of a pattern-data processor 400 including a pattern extractor.

Referring to FIG. 4A, when the pattern-data processor 400 receives a mixed signal 401, a BAR processor 410 removes a boundary artifact.

Further, a PCA processor 411 of the pattern-data processor 400 extracts PCA pattern data 402 corresponding to a principal component of voltage of the signal from which the boundary artifact is removed.

Meanwhile, the pattern-data processor 400 extracts and outputs the PCA pattern data 402 as data associated with the breathing component.

Further, an L-curve detector 412 of the pattern-data processor 400 extracts L-curve data from the PCA pattern data 402.

Further, an ICA processor 413 of the pattern-data processor 400 detects ICA component data corresponding to an ICA component.

Further, an ICA selector 414 of the pattern-data processor 400 may select and output ICA pattern data 403 corresponding to a specific component among the ICA components.

Further, a source comparator 415 of the pattern-data processor 400 may identify homogeneity between the PCA pattern data 402 and the ICA pattern data 403.

Ultimately, the pattern-data processor 400 may use the PCA pattern data 402 and the ICA pattern data 403 to respectively reconstruct the EIT data.

FIG. 4B illustrates an embodiment related to pattern data according to an embodiment of the disclosure.

Referring to FIG. 4, the frequency patterns of the mixed signal 401, the PCA pattern data 402 and the ICA pattern data 403 are illustrated.

For instance, the mixed signal 401 includes the PCA pattern data 402 and the ICA pattern data 403.

FIG. 5 illustrates an embodiment that an image is restored by an apparatus for extracting a specific component and reconstructing data according to an embodiment of the disclosure.

Referring to FIG. 5, the apparatus for extracting a specific component and reconstructing data generates EIT data 500 corresponding to a composite signal, extracts pattern data corresponding to breathing component 510, and extracts pattern data corresponding to blood flow 511.

The apparatus for extracting a specific component and reconstructing data may use the breathing component 510 to restore image data 520.

Further, the apparatus for extracting a specific component and reconstructing data may restore image data 521 based on the blood flow 511.

In other words, the apparatus for extracting a specific component and reconstructing data may separate components associated with change in air inside a lung or change in thoracic blood flow from EIT measurement data, and use the separated EIT data to individually restore images based on the change in air inside the lung or the change in thoracic blood flow.

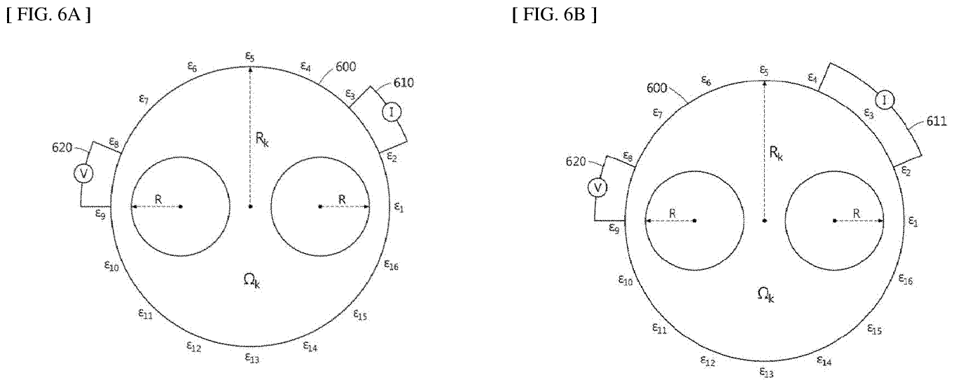

FIGS. 6A and 6B illustrate embodiments related to measurement of electric properties according to an embodiment of the disclosure.

Referring to FIG. 6A, an electrode unit 600 including a plurality of electrodes may be attached to a circumference of a subject.

For example, the plurality of electrodes may include a first electrode ε₁ to a sixteenth electrode ε₁₆.

For instance, the apparatus for extracting a specific component and reconstructing data injects current to a supplying electrode pair 610, and measures voltage through a measuring electrode pair 620.

The supplying electrode pair 610 may include a second electrode ε₂ and a third electrode ε₃ which neighbor on each other.

The measuring electrode pair 620 may include an eighth electrode ε₈ and a ninth electrode ε₉ which neighbor on each other.

Referring to FIG. 6B, the electrode unit 600 including the plurality of electrodes may be attached to the circumference of the subject.

For instance, the apparatus for extracting a specific component and reconstructing data injects current to a supplying electrode pair 611, and measures voltage through the measuring electrode pair 620.

The supplying electrode pair 611 may include a second electrode ε₂ and a fourth electrode ε₄ which do not neighbor on each other.

Here, the supplying electrode pair 611 may include the fourth electrode ε₄ skipping over the third electrodes ε3.

The measuring electrode pair 620 may also include the eighth electrode ε₈ and the ninth electrode ε₉ which neighbor on each other.

Meanwhile, the maximum and minimum values of the induced voltage may be varied depending on methods of injecting the current.

In a “0-skip” method of injecting the current between two neighboring electrodes, internal current distribution is focused between the injecting electrodes, and therefore the internal voltage may rapidly decrease as a distance from the current injecting electrode increases.

In a “1-skip” method of selecting not the immediate neighbor electrode but the next neighboring electrode, a distance between two electrodes for injecting the current may increase.

Then, the internal current spatially spreads out and slows down to such an extent that the voltage decreases.

When all the conditions of one patient are the same, “1-skip” is increased in the maximum value and the minimum value as compared with “0-skip”. In this case, the increment of the maximum value is larger than the increment of the minimum value, and therefore difference between the maximum value and the minimum value, i.e. the range of voltage may relatively increase.

Like this, when the range of the voltage increases, a preset number of voltages distinguishable from noise increases, thereby improving the quality of the image.

When the distance between two electrodes for injecting the current further increases like “2-skip,” “3-skip” or the like, it may be more effective in increasing the range of the voltage.

This is because the internal current more spreads out when the distance between two electrodes for injecting the current increases.

Meanwhile, when conductivity on a specific portion is changed, high sensitivity on this change is advantageous to make a high-resolution image based on the change. In other words, “0-skip” may be the most advantageous in terms of a space resolution.

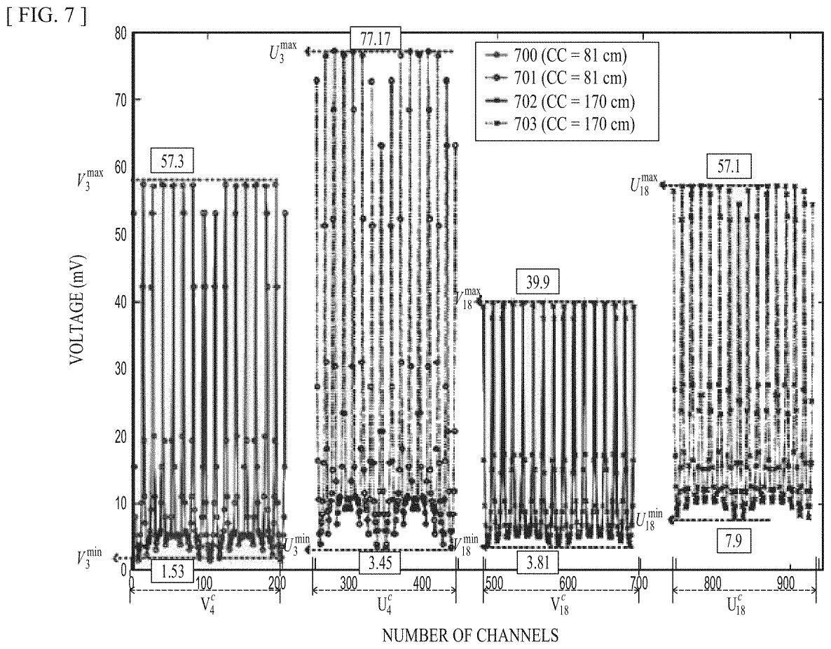

FIG. 7 illustrates an embodiment of changing a current injecting pair according to a measured circumference of a subject.

In more detail, FIG. 7 illustrates that the number of channels and the voltage are varied depending on change in the circumference of the subject and change in combination of a supplying electrode pair.

Data 700 shows that electrodes combined to form the supplying electrode pair neighbor on each other when a subject has a circumference of 81 cm.

Data 701 shows that one electrode is excluded between electrodes combined to form the supplying electrode pair when a subject has a circumference of 81 cm.

Data 702 shows that electrodes combined to form the supplying electrode pair neighbor on each other when a subject has a circumference of 170 cm.

Data 703 shows that one electrode is excluded between electrodes combined to form the supplying electrode pair when a subject has a circumference of 170 cm.

In other words, when one electrode is excluded between the electrodes combined to form the supplying electrode pair, the number of channels and the voltage measurement range may increase.

Further, when the circumference of the subject increases, the number of channels may increase and the voltage measurement range may decrease.

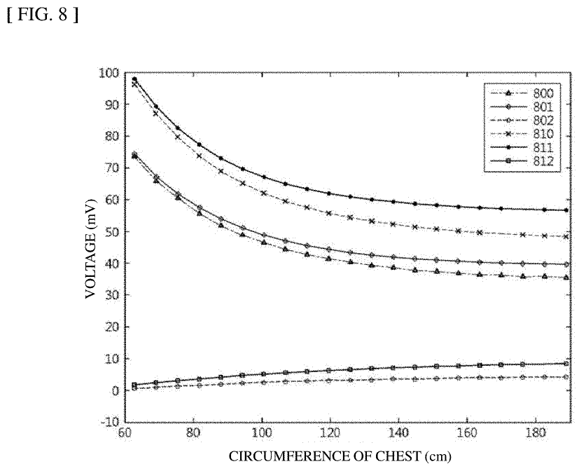

FIG. 8 illustrates an embodiment that combination of electrodes forming the current injecting pair is varied depending on a measured circumference of a subject.

Referring to FIG. 8, data 800 may correspond to a voltage measurement range obtained by excluding the minimum value from the maximum value when neighboring electrodes are combined to form the supplying electrode pair.

Data 801 may correspond to the maximum value when neighboring electrodes are combined to form the supplying electrode pair.

Data 802 may correspond to the minimum value when neighboring electrodes are combined to form the supplying electrode pair.

Data 810 may correspond to the voltage measurement range obtained by excluding the minimum value from the maximum value when one electrode is excluded between electrodes combined to form the supplying electrode pair.

Data 811 may correspond to the maximum value when one electrode is excluded between electrodes combined to form the supplying electrode pair.

Data 812 may correspond to the minimum value when one electrode is excluded between electrodes combined to form the supplying electrode pair.

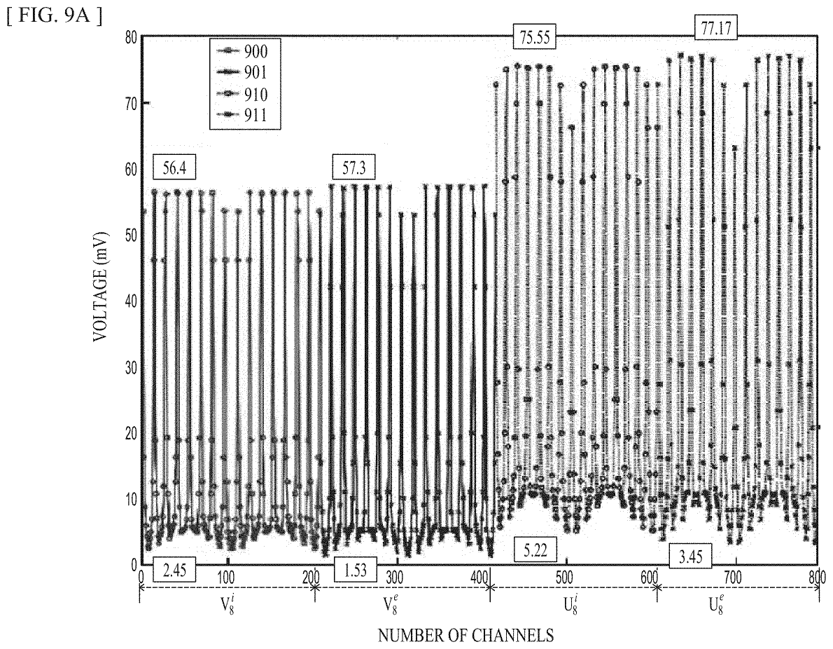

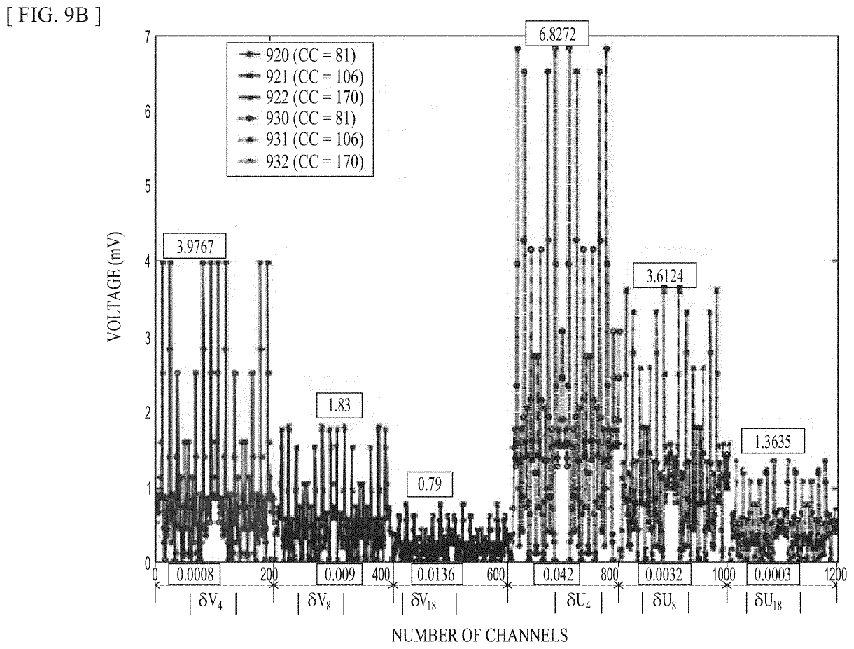

FIG. 9A illustrates an embodiment that combination of electrodes forming the current injecting pair is varied depending on a measured circumference of a subject.

In more detail, FIG. 9A illustrates the number of channels and the voltage measured based on a subject's breathing and change in combination of the supplying electrode pair.

Referring to FIG. 9A, data 900 may correspond to inspiration when neighboring electrodes are combined to form the supplying electrode pair.

Data 901 may correspond to expiration when neighboring electrodes are combined to form the supplying electrode pair.

Data 910 may correspond to inspiration when one electrode is excluded between electrodes combined to form the supplying electrode pair.

Data 911 may correspond to expiration when one electrode is excluded between electrodes combined to form the supplying electrode pair.

FIG. 9B illustrates an embodiment that combination of electrodes forming the current injecting pair is varied depending on a measured circumference of a subject.

In more detail, FIG. 9B illustrates the number of channels and the voltage measured based on change in the measured circumference of the subject and change in combination of the supplying electrode pair.

Data 920 shows that electrodes combined to form the supplying electrode pair neighbor on each other when a subject has a circumference of 81 cm.

Data 921 shows that electrodes combined to form the supplying electrode pair neighbor on each other when a subject has a circumference of 106 cm.

Data 922 shows that electrodes combined to form the supplying electrode pair neighbor on each other when a subject has a circumference of 170 cm.

Data 930 shows that one electrode is excluded between electrodes combined to form the supplying electrode pair when a subject has a circumference of 81 cm.

Data 931 shows that one electrode is excluded between electrodes combined to form the supplying electrode pair when a subject has a circumference of 106 cm.

Data 932 shows that one electrode is excluded between electrodes combined to form the supplying electrode pair when a subject has a circumference of 170 cm.

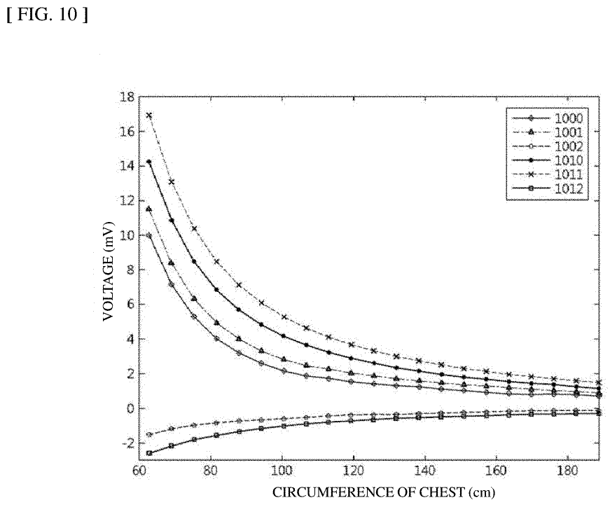

FIG. 10 illustrates an embodiment that combination of electrodes forming the current injecting pair is varied depending on a measured circumference of a subject.

Referring to FIG. 10, data 1000 may correspond to the voltage measurement range obtained by excluding the minimum value from the maximum value when neighboring electrodes are combined to form the supplying electrode pair.

Data 1001 may correspond to the maximum value when neighboring electrodes are combined to form the supplying electrode pair.

Data 1002 may correspond to the minimum value when neighboring electrodes are combined to form the supplying electrode pair.

For example, when neighboring electrodes are combined to form the supplying electrode pair, it may also be called “0-skip.”

Data 1010 may correspond to the voltage measurement range obtained by excluding the minimum value from the maximum value when one electrode is excluded between electrodes combined to form the supplying electrode pair.

Data 1011 may correspond to the maximum value when one electrode is excluded between electrodes combined to form the supplying electrode pair.

Data 1012 may correspond to the minimum value when one electrode is excluded between electrodes combined to form the supplying electrode pair.

For example, when one electrode is excluded between electrodes combined to form the supplying electrode pair, it may also be called “1-skip.”

According to an embodiment of the disclosure, the apparatus for measuring the electric properties measures a circumference of a patient's portion to which the electrodes are attached.

Next, the apparatus for measuring electric properties may estimate the maximum value, the minimum value, and the range (i.e. the maximum value−the minimum value) from the graph or table given based on the measured circumference. Here, the apparatus for measuring the electric properties employs “0-skip.”

The apparatus for measuring electric properties may identify an amplifier gain so that the product of the maximum value of the voltage and the amplifier gain can be equal to the maximum input voltage of ADC used in the system.

Alternatively, the apparatus for measuring electric properties may calculate the voltage range corresponding to the measured circumference (i.e. the maximum value−the minimum value) from the graph or table when the amplifier gain is used.

The apparatus for measuring electric properties may calculate the number of distinguishable voltages by dividing the foregoing voltage range by the noise level of the using EIT system.

The apparatus for measuring electric properties employs “1-skip” when there is a need of increasing the number of distinguishable voltages. However, the apparatus for measuring electric properties may employ “2-skip” when the number of distinguishable voltages is insufficient even though “1-skip” is used.

Meanwhile, the apparatus for measuring electric properties may repeat the foregoing process to identify the minimal “skip” by which the number of distinguishable voltages is secured.

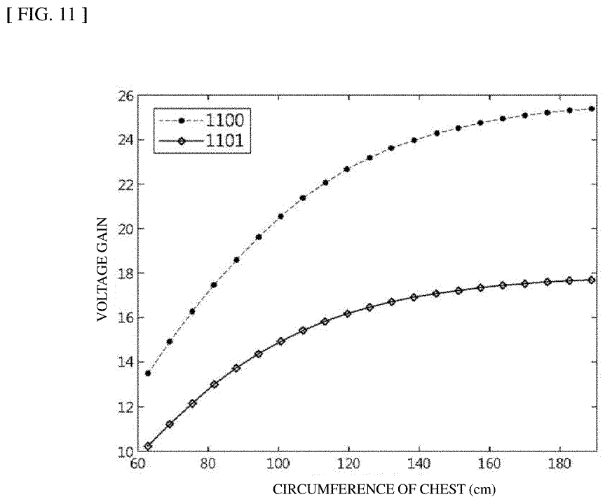

FIG. 11 illustrates an embodiment that combination of electrodes forming the current injecting pair is varied depending on a measured circumference of a subject.

In more detail, FIG. 11 illustrates a voltage gain for adjusting the maximum value of the voltage to the maximum input voltage of the ADC through “0-skip” and “1-skip.”

Data 1100 may correspond to a case that neighboring electrodes are combined to form the supplying electrode pair.

Data 1101 may correspond to a case that one electrode is excluded between electrodes combined to form the supplying electrode pair.

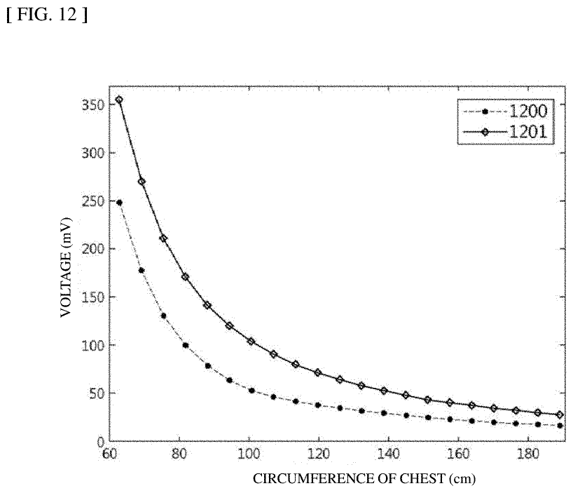

FIG. 12 illustrates an embodiment that combination of electrodes forming the current injecting pair is varied depending on a measured circumference of a subject.

In more detail, FIG. 12 illustrates that the number of distinguishable voltages is varied as the circumference increases in “0-skip” and “1-skip.”

Data 1200 may correspond to a case that neighboring electrodes are combined to form the supplying electrode pair.

Data 1201 may correspond to a case that one electrode is excluded between electrodes combined to form the supplying electrode pair.

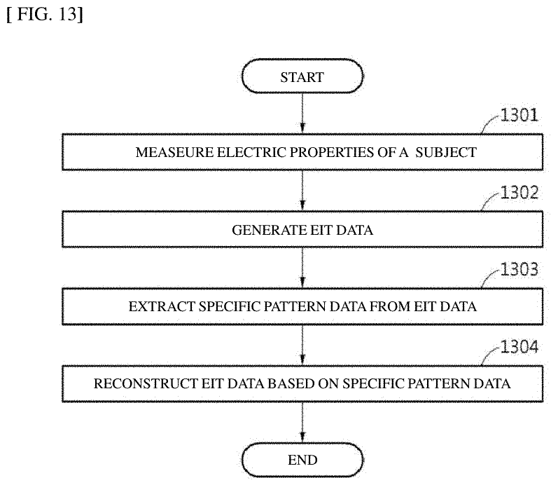

FIG. 13 is a flowchart related to a method of extracting a specific component and reconstructing data according to an embodiment of the disclosure;

Referring to FIG. 13, at operation 1301 the method of extracting a specific component and reconstructing data includes measuring electric properties of a subject.

In other words, the method of extracting a specific component and reconstructing data may include measuring a plurality of electric properties of a subject according to lapse of time, through the plurality of electrodes attached to a subject.

At operation 1302 the method of extracting a specific component and reconstructing data may include generating EIT data.

In other words, the method of extracting a specific component and reconstructing data may include generating the EIT data based on change in the plurality of measured electric properties.

At operation 1303 the method of extracting a specific component and reconstructing data may extract the specific pattern data from the EIT data.

In other words, the method of extracting a specific component and reconstructing data may use one among a signal-to-noise ratio in the generated EIT-data, PCA and ICA to analyze either of energy or frequency of the generated EIT-data.

Further, the method of extracting a specific component and reconstructing data may extract specific pattern data associated with a specific component caused by specific physiological phenomenon of a subject with respect to a frequency component based on the analyzed energy or frequency.

At operation 1304 the method of extracting a specific component and reconstructing data may reconstruct the EIT data based on the specific pattern data.

In other words, the method of extracting a specific component and reconstructing data may use the extracted specific pattern data to reconstruct the previously generated EIT-data as EIT data corresponding to a specific component.

In more detail, the method of extracting a specific component and reconstructing data may employ a difference in relative voltage change level between the specific pattern data and the previously generated EIT-data to reconstruct the previously generated EIT-data as EIT data corresponding to a specific component.

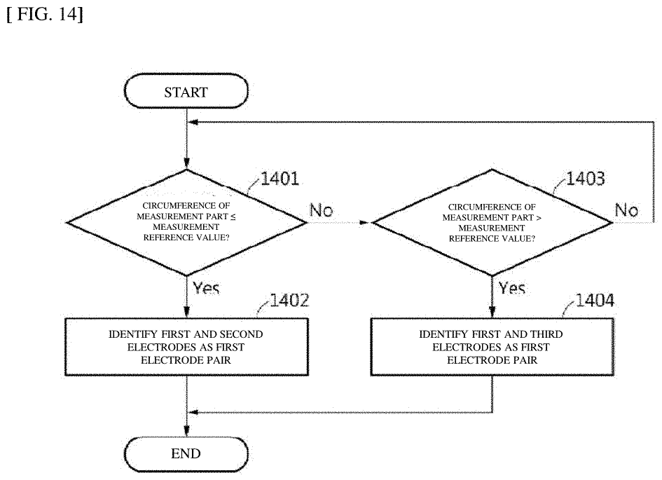

FIG. 14 is a flowchart related to a method of measuring electric properties based on EIT according to an embodiment of the disclosure.

Referring to FIG. 14, at operation 1401, the method of measuring electric properties based on the EIT includes comparing a circumference of a measurement part of a subject and a measurement reference value.

At operation 1402, the method of measuring electric properties based on the EIT may include identifying the first electrode and the second electrode as the supplying electrode pair when the circumference of the measurement part is smaller than or equal to the measurement reference value. Here, the first electrode and the second electrode may neighbor on each other.

On the other hand, the method of measuring electric properties based on the EIT enters operation 1403 when the circumference of the measurement part is greater than the measurement reference value

At operation 1403, the method of measuring electric properties based on the EIT compares the circumference of the measurement part of the subject and the measurement reference value.

At operation 1404, the method of measuring electric properties based on the EIT may identify the first electrode and the third electrode as the supplying electrode pair when the circumference of the measurement part is greater than the measurement reference value. Here, the third electrode neighbors on the second electrode, but is a little distant from the first electrode.

For instance, the method of measuring electric properties based on the EIT may further include identifying a plurality of supplying electrode pairs which are different in distance according to change in the circumference of the measurement part, by repetitively performing the operation 1402 to the operation 1404.

According to the disclosure, it is possible to extract only a component of a specific physiological phenomenon from a composite signal affected by change in internal electric properties of a human body according to a plurality of physiological phenomena.

According to the disclosure, it is possible to extract components respectively caused by change in air inside an upper airway, change in blood flow inside a carotid, movement of a neck at breathing, movement of a tongue, change in air inside a lung, or change in thoracic blood flow from electrical impedance tomography (EIT).

According to the disclosure, it is possible to restore an image based on a component associated with a specific physiological phenomenon.

According to the disclosure, it is possible to change a method of injecting current or voltage by considering the size and shape of a part to be subjected to imaging, and thus flexibly set a range of measuring the voltage or the current.

According to the disclosure, it is possible to identify an electrode pair for injecting current or voltage based on circumference of a part to be subjected to imaging, thereby flexibly setting a measurement range for voltage or current.

According to the disclosure, it is possible to improve quality of a restored image by increasing the number of voltages distinguishable from noise as measurement range for voltage or current is flexibly set.

The method according to an embodiment may be achieved in the form of a program instruction to be implemented through various computer mean, and recorded in a computer readable medium. The computer readable medium may include a program instruction, a data file, a data structure, etc. independently or combination thereof.

The program command recorded in the medium may be specially deigned or configured for embodiments, or publicly known and usable to a person having an ordinary kill in the computer software art. The computer readable recording medium may for example include magnetic media such as a hard disk, a floppy disk, and a magnetic tape;

optical media such a CD-ROM, and a DVD; magneto-optical media such a floptical disk; and a ROM, RAM, a flash memory or the like hardware device specially configured to store and implement a program command.

The program instruction may for example include not only a machine language code made by a compiler but also a high-level language code executable by a computer through an interpreter or the like. The hardware device may be configured to operate as one or more software module to perform an operation according to an embodiment, and vice versa.

Although a few embodiments have been described with exemplary examples and drawings, various changes and modifications can be made from the foregoing descriptions by those skilled in the art. For example, proper results are produced even though the described techniques are carried out in different order from that of the described method, and/or the described elements such a system, a structure, a device, a circuit, etc. are coupled or combined in a different form from that of the described method, or may be replaced or substituted with other elements or equivalents.

Therefore, equivalents to other materializations, other embodiments, and claims are also within the scope of the appended claims.

What is claimed is:

1. An apparatus for extracting a specific component based on electrical impedance tomography (EIT) and reconstructing data, the apparatus comprising:
an electrode unit comprising a plurality of electrodes to be attached to a measurement part of a subject;
an electric-property measurer configured to measure a plurality of electric properties of the subject according to lapse of time through the plurality of attached electrodes;
an EIT-data generator configured to generate EIT data based on change in the plurality of measured electric properties;
a pattern extractor configured to extract specific pattern data associated with a specific component caused by a specific physiological phenomenon of the subject from the generated EIT-data; and
an EIT-data reconstructor configured to reconstruct the generated EIT-data as EIT data corresponding to the specific component based on the extracted specific pattern data.

2. The apparatus according to claim 1, wherein the pattern extractor analyzes one of energy and frequency of the generated EIT-data through one of a signal-to-noise ratio in the generated EIT-data, principal component analysis (PCA) or independent component analysis (ICA), and extracts specific pattern data associated with a specific component caused by a specific physiological phenomenon of the subject with respect to a frequency component based on the analyzed energy or frequency.

3. The apparatus according to claim 1, wherein the EIT-data reconstructor reconstructs the generated EIT-data as EIT data corresponding to the specific component, based on difference in relative voltage change level between the extracted specific pattern data and the generated EIT-data.

4. The apparatus according to claim 1, further comprising an image restorer configured to restore an image associated with the specific component based on the reconstructed EIT data.

5. The apparatus according to claim 1, wherein the plurality of measured electric properties comprises conductivity and dielectric constant distribution.

6. The apparatus according to claim 1, wherein the specific component comprises at least one of change in air inside a lung or airway of the subject, change in blood flow inside a body, change in a component inside the body, and change in movement of a part in the body.

7. An apparatus for measuring electric properties based on electrical impedance tomography (EIT), the apparatus comprising:
an electrode unit comprising a plurality of electrodes to be attached to a measurement part of a subject; and
an electric-property measurer configured to measure a plurality of electric properties of the subject through the plurality of attached electrodes,
the electric-property measurer identifying neighboring first and second electrodes among the plurality of electrodes as a supplying electrode pair when a circumference of the measurement part is smaller than or equal to a measurement reference value, identifying the first electrode and a third electrode positioned farther away from the first electrode than the second electrode as the supplying electrode pair when the circumference of the measurement part is greater than the measurement reference value, and identifying a plurality of supplying electrode pairs which are different in distance according to change in the circumference of the measurement part.

8. The apparatus according to claim 7, wherein the electric-property measurer supplies current or voltage to the identified supplying electrode pair, and measures current or voltage induced from the current or voltage supplied through the plurality of attached electrodes.

9. The apparatus according to claim 7, wherein the electric-property measurer changes a voltage gain or a current gain according to the circumference of the measurement part when the current or voltage induced from the supplied current or voltage.

10. A method of extracting a specific component and reconstructing data based on electrical impedance tomography (EIT), the method comprising:
by an electric-property measurer, measuring a plurality of electric properties of a subject according to lapse of time through a plurality of attached electrodes attached to a measurement part of the subject;
by an EIT-data generator, generating EIT data based on change in the plurality of measured electric properties;
by a pattern extractor, extracting specific pattern data associated with a specific component caused by a specific physiological phenomenon of the subject from the generated EIT-data; and
by an EIT-data reconstructor, reconstructing the generated EIT-data as EIT data corresponding to the specific component based on the extracted specific pattern data.

11. The method according to claim 10, wherein the extraction of specific pattern data associated with a specific component caused by a specific physiological phenomenon of the subject from the generated EIT-data comprises
analyzing one of energy and frequency of the generated EIT-data through one of a signal-to-noise ratio in the generated EIT-data, principal component analysis (PCA) and independent component analysis (ICA); and
extracting specific pattern data associated with a specific component caused by a specific physiological phenomenon of the subject with respect to a frequency component based on the analyzed energy or frequency.

12. The method according to claim 10, wherein the reconstruction of the generated EIT-data as EIT data corresponding to the specific component based on the extracted specific pattern data comprises
reconstructing the generated EIT-data as EIT data corresponding to the specific component, based on difference in relative voltage change level between the extracted specific pattern data and the generated EIT-data.

13. A method of measuring electric properties based on electrical impedance tomography (EIT), in which the plurality of electric properties of a subject is measured through a plurality of electrodes to be attached to a measurement part of the subject, the method comprising:
by an electric-property measurer, identifying neighboring first and second electrodes among the plurality of electrodes as a supplying electrode pair when a circumference of the measurement part is smaller than or equal to a measurement reference value;
by an electric-property measurer, identifying the first electrode and a third electrode positioned farther away from the first electrode than the second electrode as the supplying electrode pair when the circumference of the measurement part is greater than the measurement reference value; and
by an electric-property measurer, identifying a plurality of supplying electrode pairs which are different in distance according to change in the circumference of the measurement part.

14. The method according to claim 13, further comprising
by the electric-property measurer, supplying current or voltage to the identified supplying electrode pair;
measuring current or voltage induced from the current or voltage supplied through the plurality of attached electrodes; and
by the electric-property measurer, changing a voltage gain or a current gain according to the circumference of the measurement part when the current or voltage induced from the supplied current or voltage.
